(19) United States
(12) Patent Application Publication
(10) Pub. No.: US 2007/0154468 A1
(43) Pub. Date: Jul. 5, 2007

(54) Use of CD28-specific monoclonal antibodies for producing a pharmaceutical composition for treating virus infections

(76) Inventor: Thomas Huenig, Wuerzburg (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/702,560

(22) Filed: Feb. 6, 2007

Related U.S. Application Data
(63) Continuation of application No. 10/049,464, filed on Jun. 18, 2002, now abandoned.

(30) Foreign Application Priority Data
Aug. 13, 1999 (DE) 199 39 653.1

(51) Int. Cl. A61K 39/395; C12N 5/06; C12N 5/16

(52) U.S. Cl. 424/130.1; 435/345

(57) ABSTRACT

The invention teaches the application of monoclonal antibodies being specific for CD28 and activating T lymphocytes of several to all sub-groups without occupying an antigen receptor of the T lymphocytes and thus in an antigen-unspecific manner, or an analogue thereto, for producing a pharmaceutical composition in an embodiment as a preparation or preparation package for treating virus infections of the human body or of the body of a lower warm-blooded animal with infected T lymphocytes, and wherein the pharmaceutical composition further contains a virus inhibitor, a corresponding pharmaceutical composition and a treatment plan using the pharmaceutical composition.

### FIELD OF THE INVENTION

The invention relates to the application of monoclonal antibodies being specific for CD28 and activating T lymphocytes of several to all sub-groups without occupying an antigen receptor of the T lymphocytes and thus in an antigen-unspecific manner, or an analogue thereto, for producing a pharmaceutical composition in an embodiment as a preparation or preparation package for treating virus infections of the human body or the body of a lower warm-blooded animal with infected T lymphocytes, to a package component with such antibodies or analogues, to a method for treating virus infections under application of such a composition and to a treatment plan under application of such a composition.

### BACKGROUND OF THE INVENTION

The HIV undergoes a life cycle, wherein it may exist in different latency stages. A first latency stage is called pre-integrative, which means that the HIV has been imported in the host cell and submitted at least in part to the reverse transcription, however not integrated yet in the cell core as a pro-virus. This pre-integrative latency stage may remain latently functional over a multitude of weeks until the loss of the functionality. The pre-integrative latency requires that the host cell is at rest. Another latency stage is designated post-integrative, which means that the HIV as a pro-virus has been integrated in the cell core, the host cell however being at rest, for instance by de-activation, and thus no virus replication taking place. The post-integrative latency is rather stable in time and continues to be effective until an activation of the host cell. An activation of latent (post or pre-integrative) HIV containing host cells takes place by various stimuli, with the consequence of the activation of the virus replication, too, the host cell being destroyed and the virus being released in the body liquid. The above explanations apply in principle to all retro viruses. A virus in a latent stage will be called a latency virus in the following.

Previous therapy approaches with reverse transcriptase inhibitors and possibly protease inhibitors suppress the virus multiplication after activation of the latency virus. Thus free HIV under therapy will disappear from the circulation, leukocytes at rest however will continue containing latency viruses (T. W. Chun et al., Proc. Natl. Acad. Sci. USA, 94:13193-13197 (1997); D. Finzi et al., Science, 278: 1295-1300 (1997); J. K. Wong et al., Science, 278:1291-1295 (1997)). The consequence is a re-appearance of replication-capable viruses when the therapy is interrupted (M. D. de Jong et al., AIDS, 11:F79-84 (1997)), and the disease goes on. The therapy with reverse transcriptase inhibitors and possibly protease inhibitors is however toxic, and a necessary lifetime treatment is therefore limited or critical. Further, the treatment with reverse transcriptase inhibitors and possibly protease inhibitors underlies high costs.

### STATE OF THE ART

As a consequence of the above basic problems it has been suggested to make accessible latent HIV of the therapy by reverse transcriptase inhibitors and possibly protease inhibitors by the simultaneous treatment with immune-stimulating agents activating the latent HIV (“flush-out”; O. J. Cohen et al., J. Am. Med. Assoc., 280:87-88 (1998); J. Cohen, Science, 279:1854-1855 (1998); D. D. Ho, Science, 280: 1866-1867 (1998); L. K. Schrager et al., J. Am. Med. Assoc., 280:67-71 (1998)). Particularly in the document T. W. Chun et al., Nature Medicine, Volume 5, Number 6, pp. 651-655 (1999) is shown that by using the T cell growth factor Interleukin-2 (IL-2) together with the HAART therapy (see below), a significant reduction of the amount of replication-competent HIV is found in the T lymphocytes at rest. However, the largest portion of CD4 T lymphocytes does not express receptors for the growth factor IL-2. These cells and thus the latent HIV contained therein cannot therefore be accessed by administering IL-2. The above basic problems will therefore persist and are at most slightly reduced. Further, the administration of IL-2 has substantial disturbing side effects further questioning the success of such a strategy.

The most common therapy under the application of reverse transcriptase inhibitors is the HAART therapy (“highly active anti-retroviral therapy”). It consists in the combined application of two reverse transcriptase inhibitors, e.g. the nucleoside analogue AZT (or zidovudine) and 3TC (or lamivudine), together with one or more protease inhibitors. An example for a protease inhibitor is IDV (indinavir). With regard to the HAART therapy, the substances used and the treatment plan, reference is made to the following documents: J. Laurence, HAART Regiments: Do the effects last? in the AIDS Reader 7(6):84-85 (1997); R. M. Gulick et al., N. Engl. J. Med., 337:734-739 (1997); S. M. Hammer et al., N. Engl. J. Med., 337: 725-733 (1997); and F. J. Jr. Palella et al., N. Engl. Med., 338:853-860 (1998). Further examples for suitable reverse transcriptase inhibitors are the nucleoside analogues d4T (stavudine), ddl (didanosine) and ddc (zalcitabine) as well as the non-nucleoside analogues DLV (delavirdine) and NVP (nevirapine). Further examples for suitable protease inhibitors are NFV (nelfinavir), RTV (ritonavir) and SQV (saquinavir).

From document WO 98/54225 monoclonal antibodies well tolerated for human beings and being specific for human CD28 and activating human T lymphocytes of several or all sub-groups without occupying an antigen receptor of the human T lymphocytes and thus in an antigen-unspecific manner are known in the art. With regard to further background information, reference is made to the citations listed in this document. It is also known from this document to use these monoclonal antibodies for treating diseases with pathologically reduced CD4 T cell counts, as for instance AIDS. The background of this application is that by means of these antibodies the CD4 T cell counts can be increased again. An association with the administration of reverse transcriptase inhibitors and possibly protease inhibitors is not claimed. The document WO 98/54225 is therefore explicitly and to full extent referred to.

### OBJECT OF THE INVENTION

With regard to the closest state of the art according to T. W. Chun et al., Nature Medicine, Volume 5, Number 6, pp. 651-655, it is the object of the invention to develop a pharmaceutical composition or a treatment plan, by means of which on one hand at least the largest part of the HIV latency viruses, if not all of them, are activated (and thus made inhibitable by virus inhibitors and finally destroyable), and on the other hand the side effects are reduced.

### BASIC PRINCIPLES OF THE INVENTION

The invention teaches the application of monoclonal antibodies being specific for CD28 and activating T lymphocytes of several to all sub-groups without occupying an antigen receptor of the T lymphocytes and thus in an antigen-unspecific manner, or an analogue thereto, for producing a pharmaceutical composition in an embodiment as a preparation or preparation package for treating virus infections of the human body or the body of lower a warm-blooded animal with infected T lymphocytes, wherein the pharmaceutical composition further contains a virus inhibitor. It is understood that the drug components are applied in pharmaceutically effective doses.

The invention further teaches a pharmaceutical composition in an embodiment as a preparation or preparation package, with pharmaceutically effective doses of the drug components: a) preferably monoclonal antibodies well tolerated for human beings, preferably being specific for CD28, preferably human CD28 and activating T lymphocytes, preferably human T lymphocytes, of several to all sub-groups without occupying an antigen receptor of the T lymphocytes and thus in an antigen-unspecific manner, or an analogue thereto; b) a virus inhibitor, preferably a reverse transcriptase inhibitor, c) as an option a reverse transcriptase inhibitor being different from b); d) as an option a protease inhibitor; e) as an option a protease inhibitor different from d). In addition to the drug components named above, further drugs and/or substances being suitable or necessary for the galenic preparation may be contained.

The invention further teaches a package component of a preparation package containing the drug component a).

The invention further teaches a method for treating virus infections with lentivirus, in particular HIV, wherein a pharmaceutical composition is administered to a human body or the body of a lower warm-blooded animal attacked by the infection, or a method for treating virus infections with lentivirus, in particular HIV, wherein are administered to a human body or to the body of a lower warm-blooded animal the following drug components in pharmaceutically effective doses: a) preferably monoclonal antibodies well tolerated for human beings and being specific for CD28, preferably human CD28 and activating T lymphocytes, preferably human T lymphocytes of several or all sub-groups without occupying an antigen receptor of the human T lymphocytes and thus in an antigen-unspecific manner, or an analogue thereto, b) a virus inhibitor, preferably a reverse transcriptase inhibitor, c) as an option a reverse transcriptase inhibitor different from b), d) as an option a protease inhibitor, e) as an option a protease inhibitor different from d).

The invention further teaches an application of a preferably monoclonal antibody a) well tolerated for human beings and being specific for CD28, preferably human CD28 and activating T lymphocytes, preferably human T lymphocytes of several or all sub-groups without occupying an antigen receptor of the T lymphocytes and thus in an antigen-unspecific manner, or an analogue thereto, and a virus inhibitor b), preferably a reverse transcriptase inhibitor in the form of a mixture or of spatially separated compositions, one of those containing the drug component a) and the other one containing the drug component b), as a means for application during the treatment of virus infections with lentiviruses, in particular AIDS, in human beings or lower warm-blooded animals according to a treatment plan comprising one or more cycles, the treatment plan comprising the following steps: i) first the drug component b) is continuously administered in a pharmaceutically effective dose, ii) after a given time duration of step i) the drug component a) is administered in a pharmaceutically effective dose, with continued administration of the drug component b), iii) as an option step ii) will be repeated once or several times after a given break period, with continued administration of the drug component b). In this application, an administration of the drug component a) may be provided prior to step i).

The invention is firstly based on the finding that by the activation of a large part of the T lymphocytes, viruses at rest (latency viruses) are also activated and are thus made destroyable by virus inhibitors, in particular reverse transcriptase inhibitors (and/or protease inhibitors). Further, the invention is based on the surprising finding that the parallel application of (toxic) reverse transcriptase inhibitors, for instance of the (toxic) HAART therapy, does not disturb the activation of the T lymphocytes. This has, as a result, a double meaning and therefore a synergistic effect. On one hand, the activation of a large part of the T lymphocytes is secured, in spite of the parallel HAART therapy, with the consequence of the destruction of practically the complete latency virus reservoir by the HAART therapy. On the other hand, simultaneously the anyway pathologically reduced number of T lymphocytes is increased again, with the consequence of a stabilization of the immune system. Further, by the means according to the invention, presumably non-T cell reservoirs for latency viruses (for instance macrophages) are also activated indirectly by the strong general stimulation of the immune system or of the T cells with the corresponding cykotine release, and thus these viruses can also be activated and destroyed, another synergy.

At last it is achieved that not only the free virus is virtually completely eliminated, but also virtually the complete latency virus reservoir is made accessible by activation of the destruction. Therefore the HAART therapy needs not be performed for the whole life, at least however only in very large time intervals. Another surprising advantage over the closest state of the art is that the antibodies used according to the invention do not seem to generate any side effects, as could be seen from animal tests. In total, a substantially more effective elimination of the virus is achieved, together with a clearly better feeling of the patient already during the therapy.

In this context it may be noted that with the monoclonal antibodies used according to the invention actually all CD4 T cells can be excited to proliferation. Only a sub-population of CD8 T lymphocytes not expressing CD28 cannot be activated by activating CD28-specific reagents. However, this is not a substantial reservoir of HIV, since the primary receptor for the HIV is the CD4 molecule.

### DEFINITIONS

As monoclonal antibodies are designated antibodies being produced by hybrid cell lines (so-called hybridoms) having in turn been generated by fusion of a B cell producing antibodies of animal or human origin with a suitable myeloma tumor cell. In this description, the term monoclonal antibodies includes the derivatives thereof, too.

CD28 is a cell surface molecule of known amino acid sequence expressed on T lymphocytes of human or animal origin, said molecule having received the abbreviation CD28 from the Human Leukocyte Typings Workshop.

Activation of T lymphocytes means the increase of the metabolic activity, the increase of the cell volume, synthesis of immunologically important molecules and initiation of cell division (proliferation) of T lymphocytes upon an external stimulus.

For instance, these processes are initiated by the occupation of the CD28 molecule on T cells by special CD28-specific monoclonal antibodies. The activation of T lymphocytes with the described side effects is part of the physiological immune reaction, control may however be lost there in pathological situations (lymphoproliferative diseases), or the reaction may be insufficient (immunodeficiency).

Constant components of an antibody are sections not being important for antigen detection, in contrast to the variable sections defining the antigen specificity of an antibody. Constant components are however different for antibodies of different types, and consequently also for animals and human beings. The constant components of an antibody have to correspond to those of an organism to be treated with the antibodies, in order to be well tolerated.

Derivatives of monoclonal antibodies are modifications of the monoclonal antibody having been generated by conventional biochemical or gene manipulations. This is for instance the case for the humanization of a monoclonal antibody of the mouse by partial replacement of structural (constant) components of the mouse antibody by those of a human antibody. Derivatives are further monoclonal antibodies being chemically modified, nevertheless however fulfilling the functions described in this invention. A common criterion is always the CD28-specificity with a stimulating effect.

Analogues are substances not being monoclonal antibodies, however fulfilling the functions described in this invention. Examples are the “tailored” highly specific synthetic proteins or RNA or DNA molecules (e.g. aptamers, in particular aptamers stabilized against nucleic acid dissociating enzymes or RNA or DNA molecules). A common criterion is always the CD28 specificity with a stimulating effect.

A determinant is the section of a molecule defined by the binding specificity of one or more antibodies.

The term therapeutically active dose designates, in conjunction with virus inhibitors, for instance reverse transcriptase inhibitors being different (as an option protease inhibitors), a dose that leads to a significant reduction of the amount of replication-capable viruses for a given time period after administration of the virus inhibitors to a patient or in a test system, compared to the amount of replication-capable viruses after the same time and with the same initial amount of replication-capable viruses, however without any administration.

The term therapeutically active dose designates, in conjunction with antibodies used according to the invention, a dose leading to a significant increase of the CD4 T cell counts and/or expression of serologically detectable activation markers (CD25, CD45R0, CD71) after a defined period of time in an organism or test system to which the antibodies were administered, compared to the respective values after the same period of time and identical initial values, however without any administration.

Well tolerated for human beings are antibodies being humanized. It may be noted here that antibodies not being humanized and consequently not covered by the definition given here for antibodies well tolerated for human beings may nevertheless be used for the therapy of human beings. For the therapy of human beings thus all antibodies may be used that do not cause any immune reactions over a given period of time, as for instance detectable by determination of anti-immunoglobulin antibodies as stopping criterion.

As a virus inhibitor is designated every drug directly or indirectly inhibitingly affecting an arbitrary phase of the life cycle of a virus. In addition to reverse transcriptase inhibitors and protease inhibitors, for instance inhibitors of the cell surface receptors are also possible where a virus docks, or inhibitors of all positively regulatory proteins or processes of a virus, including inhibitors of cellular positively regulatory substances acting on the long terminal repeats of a virus. In principle, such drugs are also possible that are no inhibitors, but induce negatively regulatory proteins or processes of a virus; such drugs are also covered by the term virus inhibitor.

The term of continuous administration of a drug b) and/or c) and/or d) and/or e) means that a (per se possibly discontinuous) treatment plan to be applied to this component is executed in a continuous manner. Insofar the term continuous administration also comprises, for instance for the HAART therapy, a variation and individual adaptation of the drug or the dosage thereof following the continuous administration.

### DETAILED REPRESENTATION OF THE INVENTION

In the following, suitable or preferred embodiments of the invention are listed and described in more detail.

Preferred is the application according to the invention for producing a pharmaceutical composition for treating diseases, wherein CD4 T lymphocytes, in particular human CD4 T lymphocytes, are infected, and wherein the monoclonal antibodies are specific for human CD28 and the monoclonal antibodies are as an option well tolerated for human beings. When treating infections of human CD4 T lymphocytes, that is a human being, the antibodies have to be specific for human CD28, not necessarily however well tolerated for human beings. The invention can for instance be used when the virus infection is an infection caused by retrovirus, in particular lentivirus, for instance HIV.

It is suitable if the virus inhibitor is a reverse transcriptase inhibitor, preferably a pyrimidin nucleoside analogue, most preferably 3′-azido-3′-desoxythymidine (AZT or zidovudine), and as an option further other nucleoside analogues different therefrom, preferably 3TC, are included in the pharmaceutical composition. The pharmaceutical composition may in addition contain a protease inhibitor and as an option further additional other protease inhibitors different therefrom. With a drug combination of at least two reverse transcriptase inhibitors and as an option at least one protease inhibitor operates the HAART therapy.

Monoclonal antibodies used according to the invention are available in various ways. A suitable version according to the embodiments is available by A) a generation of hybridom cells capable of producing monoclonal human CD28-specific animal antibodies by an immunization with non-T tumor cell lines on which human CD28 is expressed, B) if applicable, a humanization of the monoclonal animal antibodies available from hybridom cells according to step A by biochemical or gene-technological exchange of constant components of the animal antibodies by analogous constant components of a human antibody or exchange of genes of the hybridom cells corresponding to the components, C) secernation of the monoclonal antibodies in hybridom cultures and isolation of the monoclonal antibodies therefrom or production of the monoclonal antibodies by injection of the hybridom cells into animals, for instance mice, and isolation of the monoclonal antibodies from the body liquid of the animals. The hybridom cells suitable for the production of monoclonal animal antibodies specific for human CD28 are available by a) a generation of a plasmid by means of insertion of human CD28 cDNA into the pHβApr-1-neo vector after excision of the SalI-HindIII fragment and production of protoplasts from Escherichia coli (MC1061) carrying the plasmid, b) a fusion of the protoplasts with mouse A20J and/or L929 tumor cells by means of polyethylene glycol, c) a cultivation of the cells obtained in step b, d) a screening of the transfected mouse A20J and/or L929 cells on the expression of human CD28 and selection of mouse A20J and/or L929 cells expressing human CD28, e) an immunization of BALB/c mice with the mouse A20J and/or L929 cells expressing human CD28, f) a removal of spleen cells of the mice thus immunized and a fusion of the spleen cells with cells of the cell line X63-Ag 8.653 by means of polyethylene glycol, g) a selection of the hybridom cells thus obtained such that in the remainder of selected hybridom cells there are contained antibodies binding to mouse A20J and/or L929 cells expressing human CD28, and h) a cultivation/sub-cloning of the hybridom cells obtained in step g. In place of steps a) to d) other expression systems accessible to the man skilled in the art may of course also be employed. Human CD28 is freely available from Dr. A. Aruffo and Dr. B. Seed having published the sequence and also the following document: Aruffo, A., and Seed, B., 1987, “Molecular cloning of a CD28 cDNA by a high efficiency COS cell expression system”, Proc. Natl. Acad. Sci. USA, 84:8573. From this document can therefore be taken details about the production of the human CD28 cDNA. Further, every man skilled in the art may very easily and quickly produce a human CD28 cDNA clone by means of the sequence laid down in the gene bank and of the polymerase chain reaction. The pHβApr-1-neo vector is freely available from the authors of the document Gunning, P. et al., 1987, “A human β-actin expression vector system directs high-level accumulation of antisense transcripts”, Proc. Natl. Acad. Sci. USA, 84:4831. “neo” is used for neomycin resistance. The step c) is performed therefore in presence of neomycin. The above cell lines and/or micro-organisms are freely available and commercially obtainable from the American Type Culture Collection (ATCC). With regard to Escherichia coli (MC1061) reference is made to the document Meissner, P. S., et al., 1987, “Bacteriophage gamma cloning system for the construction of directional cDNA libraries”, Proc. Natl. Acad. Sci. USA, 84:4171.

The basic approach for the production of hybridom cells, the humanization and the generation of the monoclonal antibodies from (humanized) hybridom cells is well known to the man skilled in the art and needs not be explained here in more detail. Basically, all cell lines, in particular the cell lines being usual for the production of hybridom cells, being known and being freely accessible, can be used. For the production of the monoclonal antibodies, beside the approach described below, in principle the recombinant expression well known to the man skilled in the art is applicable.

However, applicable monoclonal antibodies are available in other ways, too. For instance, the immunization in step A) may take place against soluble or dissolved recombinant human CD28. The humanization may be made dispensable by using, for the production of the hybridom cells, animals being modified by gene technology in such a way that the generated antibodies already comprise the human constant components. A completely different approach for the production of monoclonal antibodies is that the antigen binding domains of (for instance human) antibodies are expressed by gene technology in a highly complex bacteriophages library, wherefrom suitable binding domains are isolated by their affinity to CD28, and can be expanded to complete antibodies.

With regard to the pharmaceutical composition, the drug components b) to e) can be selected, dosed and made ready for administration corresponding to the HAART therapy. In principle, all existing and future variants of the HAART therapy or of another therapy can be used, as long as the activating effect of the monoclonal antibodies is not (over-) compensated by one or more drug components. For the existing therapy approaches it is often promising when the drug component b) is a pyrimidin nucleoside analogue, preferably 3′-azido-3′-desoxythymidine, and/or the drug component c) is 3TC, and/or the drug component d) is provided in a conclusive manner. With regard to a variant of a treatment plan according to the invention, it may be useful if the drug components b) to e) are part of a first package component, and the drug component a) is part of a second package component.

When using the invention in a treatment method, the drug components b) to e) can be selected, dosed and administered according to the HAART therapy, the drug component a) being administered before, together with or after the drug components b) to e). In detail, the drug components b) to e) may be administered continuously and the drug component a) once or several times in intervals with breaks. The drug component b) may be a pyrimidin nucleoside analogue, preferably 3′-azido-3′-desoxythymidine, and/or the drug component c) may be 3TC, and/or the drug component d) may be provided in a conclusive manner.

For a treatment plan according to the invention, the administration of the drug component b) may take place by a treatment sub-plan being the HAART therapy. First the HAART basic therapy is performed over a period of time of 1 to 12 months, preferably 2 to 6 months, most preferably 2 to 4 months, for instance 3 months (1 month=30 days). During this time the CD4 T cell counts and/or virus load and/or latency virus can regularly be examined (for instance corresponding to the document T. W. Chun et al., Nature, 387:183-188 (1977)), and on the basis of these results the HAART therapy may be adapted individually to the patient (by selection or exchange and combination of the reverse transcriptase inhibitors and/or protease inhibitors and of the respective doses). In a first cycle, a preferably intravenous injection in a dose of 0.1 to 50, preferably 0.5 to 20, most preferably 0.5 to 5 mg/kg body weight can then be performed, while maintaining the HAART therapy. The above dose may be administered once or in 2 to 10, preferably 2 to 5 parts over a period of time of 1 hour to 1 month, preferably 1 day to 5 days, in a regular or irregular distribution. In a break following thereupon (while maintaining HAART) of 1 day to 6 months, preferably 0.5 to 2 months, for instance 1 month, a supervision of the values mentioned above for the basic therapy and/or of the blood picture and/or the blood values and/or clinical-internistic results and/or the creation of anti-immunoglobulin antibodies (anti-animal Ig for non-humanized, anti-idiotypical after humanization). After the rest break the cycle may if necessary be repeated, beginning with the administration of the antibody. When PBMC (lymphocytes and monocytes) are free from virus, latency virus and pro-virus, with the patient's agreement a lymph node biopsy may be performed for verification. In case of a positive result (positive=detection of virus or latency virus or pro-virus) further cycles of the above kind may be added. In case of a negative result, the administration of HAART drugs and of antibodies may be terminated. It is recommended to perform further control checks of the above kind in certain time intervals after termination, in order to restart the treatment if necessary. The above explanations apply in a corresponding manner to a treatment method.

For the invention can be used for instance the monoclonal antibody CMY-2, available from hybridom cells according to deposit DSM ACC2353, or the clone ANC28.1/5D10 commercially available from ALEXIS Deutschland GmbH, D-35305 Gruenberg and produced by Ancell Corporation, USA, or a preferably humanized or human-tolerated variant of the clone ANC28.1/5D10.

Monoclonal antibodies employed according to the invention may in particular have specificity for determinants of the human CDN28 molecule, which are difficult to access on the naturally expressed CD28 molecule, and the occupation thereof by the monoclonal antibodies will lead to the activation of the T cells.

The galenic preparation of drug components or of mixtures thereof for the various administration types is well known to the man skilled in the art and needs not be explained here in more detail.

Explanations given to a claim category apply in a corresponding manner to the subject matters of the other claim categories.

As an option, for the invention, additional drugs may be present or be used, such substances differing from the drugs mentioned above and used according to the basic concept of the invention. Such additional drugs are for instance drugs acting against possible side effects. Just as examples are here mentioned anti-TNF antibodies (TNF=tumor necrosis factor) in the case of pro-inflammatory TNF reaction. Additional drugs are further substances being helpful in conjunction with the expansion of the immune system. For instance, by administration of IL-2, the proliferation of CDB cells can be induced or increased. Generally, immuno-modulating drugs may be used, according to the immunological (detail or side) process that is suitably promoted (or inhibited) according to the invention. Examples here are oligonucleotides containing CpG motives (D. Klinman et al., Proc. Natl. Acad. Sci. USA, 93:2879-2883 (1996)).

In the following, the invention will be described in more detail, based on examples of execution. Among other matters, the production of monoclonal antibodies used according to the invention is explained. In these embodiments, screening methods are also made clear by means of which monoclonal antibodies according to the invention or basic hybridom cells can be selected. In the following examples, the therapeutical effects can be seen.

### EXAMPLE 1

### Production and Activity of a First Monoclonal Antibody to be Used According to the Invention

1.1 General Information.

The represented experiments and the examples about the activities of “direct” CD28-specific monoclonal antibodies have been made in the animal model of the rat, wherein as an example for a “classic” CD28-specific antibody the monoclonal antibody JJ319 is used, and as an example for a “directly” activating monoclonal antibody the monoclonal antibody JJ316. Both antibodies are freely accessible and available from Pharmingen Co., San Diego, USA. JJ319 and JJ316 are further available according to the document M. Tace et al., Immunology, 1995, 154: 5121-5127 to which reference is explicitly made here, also with regard to the production of hybridom cells and monoclonal antibodies.

1.2 Production of Monoclonal Antibodies.

In this example, the production of monoclonal antibodies according to the invention, this means human CD28-specific monoclonal antibodies, is described in more detail. These are also called CMY-2 in the following. Human CD28 from a cDNA library was expressed in an recombinant manner in A20J and/or L929 cell lines. Firstly, for this purpose, a plasmid was generated by means of insertion of human CD28 cDNA into the pHβApr-1-neo vector after excision of the SalI-HindIII fragment. From Escherichia coli (MC1061) protoplasts carrying the plasmid were produced. Then took place a fusion of the protoplasts with mouse A20J and/or L929 tumor cells by means of polyethylene glycol. The transfected cells thus obtained were cultivated in a usual manner. Then followed a screening of the transfected mouse A20J and/or L929 cells on the expression of human CD28 and selection of mouse A20J and/or L929 cells expressing human CD28.

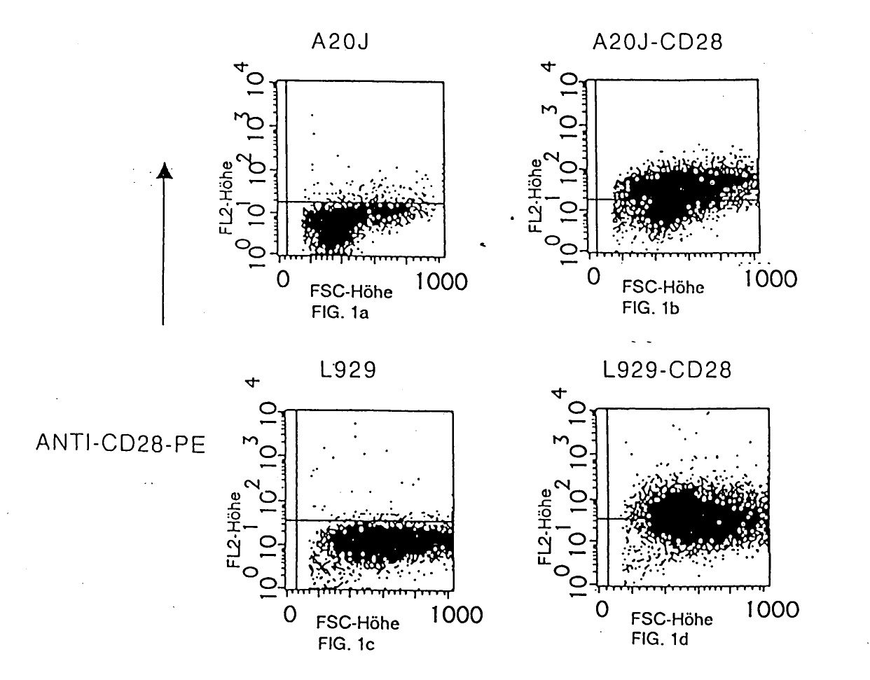

The detection of the successful expression was made by means of a conventional commercially available, fluorescence-marked antibody with specificity for human CD28 (9.3-phycoerythrin). As a negative check, non-transfected A20J and L929 cells were colored with the same antibody. The transfectants (A20J-CD28 and L929-CD28) showed a higher fluorescence intensity. Since not all cells were CD28-positive, CD28-positive cells were sub-cloned and used for immunization. As can be seen in FIG. 1 from the displacement of the dot clouds towards top in the two right-hand diagrams, these cells reacted with the commercial antibody, that means, expressed human CD28 at their surface.

The A20J human CD28 cell line was used for the immunization of BALB/c mice. Cell fusion and screening were performed as follows: i) Immunization of BALB/c mice with the mouse A20J cells expressing human CD28 (6 IP injections and then 1 IV). ii) Extraction of spleen cells of the thus immunized mice and fusion of the spleen cells with cells of the cell line X53-Ag 8.653 by means of polyethylene glycol. iii) Selection of the thus obtained hybridom cells such that in the remainder of selected hybridom cells there are contained antibodies binding to mouse A20J and/or L929 cells expressing human CD28.

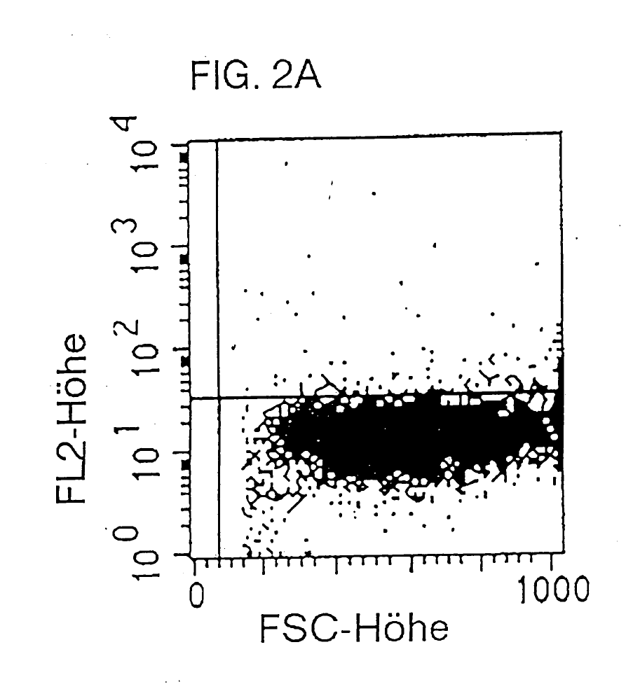

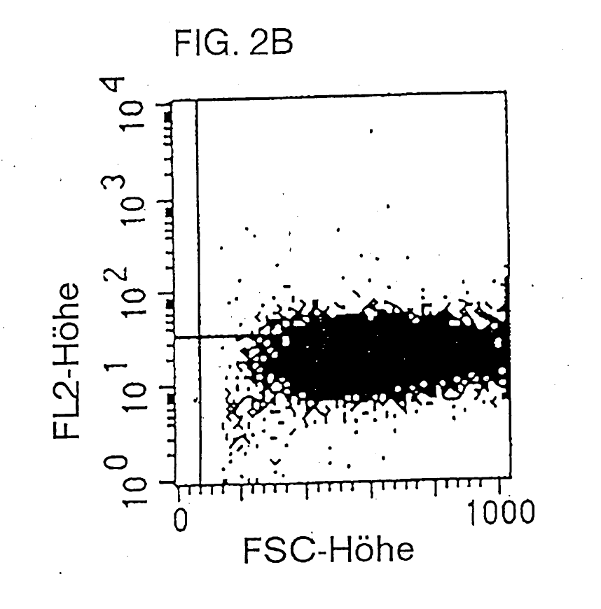

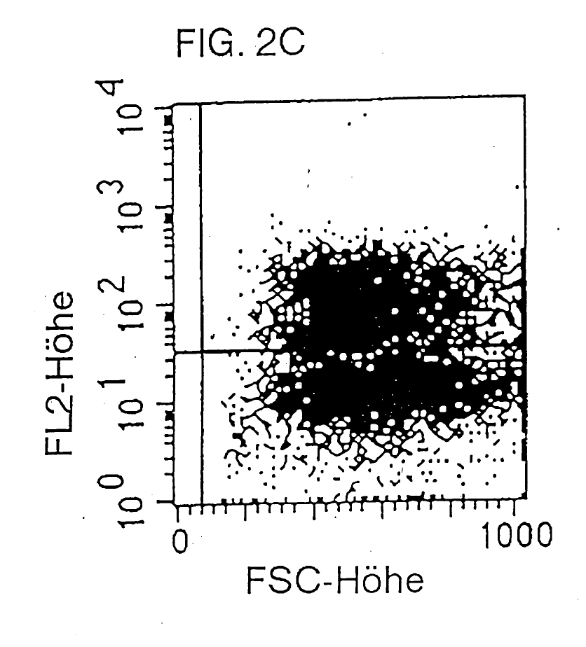

As a read-out was used the coloration of CD28 transfected and un-transfected mouse L929 tumor cells. FIG. 2 shows that thus isolated monoclonal antibody CMY-2 differentiates transfected and un-transfected cells by fluorescence intensity. The differential screening for antibodies against human CD28 takes place as follows. 50 μl remainder each of cultivated cell hybridoms were taken and incubated for 15 min with a mixture of L929 cells and L929-CD28 transfectants. After washing, the cells were colored with DaMIg-PE. Part A shows the negative check. The cells were incubated with DaMIg-PE only. Part B shows the coloration with a remainder being slightly positive, but does not show any difference between the two cells. Part C shows the cells colored with a remainder of CMY-2.

In not shown experiments, peripheral human blood cells were colored with the newly isolated CMY-2 and the “classic” CD28-specific antibody 9.3. An identical expression pattern was found on the sub-populations of human blood cells.

As a summary, the experiments show that CMY-2 is a human CD28-specific antibody.

Then CMY-2 was tested with human T lymphocytes enriched from peripheral blood to approx. 80% for classic co-stimulating and to “directly” stimulating activity. The T cell proliferation was measured by integration of ³H thymidine between the second and third day of the culture. The following results were obtained:

Costimulation:

| | | |
|---|---|---|
| Unstimulated cells | 276 | cpm |
| CD3-specific antibodies | 3,111 | cpm |
| CD3-specific antibodies + CMY-2 | 51,676 | cpm |

Direct stimulation:

| | | |
|---|---|---|
| Solid phase anti-mouse Ig | 379 | cpm |
| Solid phase anti-mouse Ig + control-mAb | 258 | cpm |
| Solid phase anti-mouse Ig + plus CMY-2 | 19,115 | cpm |

For explanation: Anti-CD3 causes T cell receptor stimulation (CD3 is part of the TCR complex). CMY-2 was used in the form of a not purified culture remainder (50*final volume). According to experiences, the effective mAb concentration to be expected is sub-optimum for a direct activation, however sufficient for the costimulation. The experiment shows that CMY-2 has directly activating properties.

Hybridom cells producing CMY-2 have been deposited at the DSMZ, Deutsche Sammlung von Mikroorganismen und Zellkulturen GmbH, Mascheroder Weg 1b, D-38124 Braunschweig, under number DSM ACC2353 (20 May 1998).

1.3 Proliferative Response to the Antibody of 1.2.

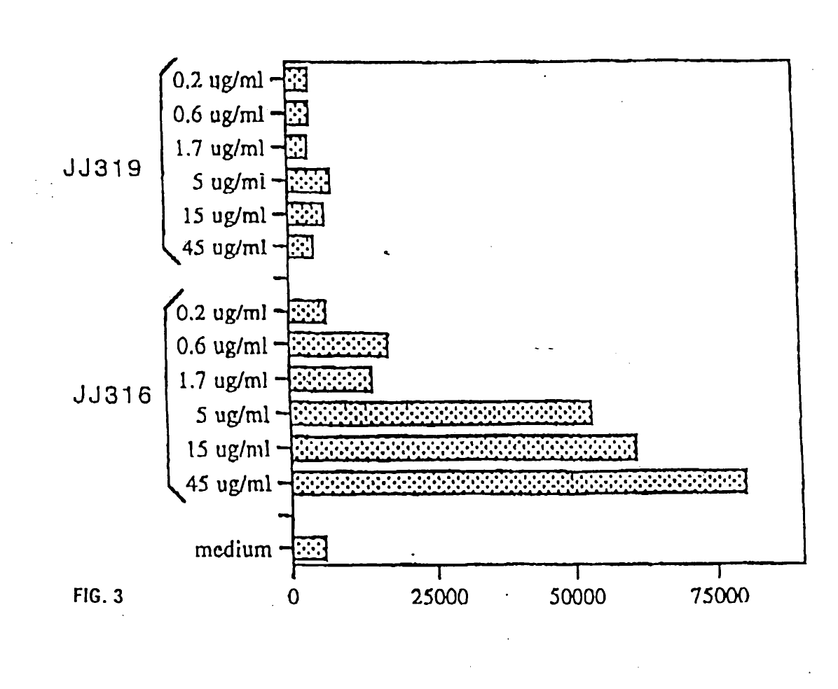

FIG. 3 shows the proliferative response to unseparated lymph node cells of the rat to the “directly” stimulating CD28 specific monoclonal antibody (JJ316) and the absence of such a response when using a “classic” CD28-specific monoclonal antibody (JJ319). The cells were cultivated for two days in 0.2 ml medium (RPMI 1640, available from GIBCO/BRL, containing 5% FCS [fetal calf serum] in presence and absence of the mentioned additions at a density of 1 million cells per ml in a gassed incubator. The cell division activity was determined by integration of radioactive-marked thymidine (1 μCi/sample for 16 hours, 1 Ci=37 GBq, determination with β detector).

In contrast to published results (Stiefken et al., Cellular Immunology, 1997, 176:59-65), this result shows that it is not necessary for the T cell activation by directly activating CD28-specific monoclonal antibodies to artificially cross-link these by a second antibody. Rather, the presence of non-T cells from lymphoid organs, namely of B lymphocytes and so-called accessory cells is sufficient to permit a direct activation by CD28-specific monoclonal antibodies added in a dissolved state. Probably, this takes place by binding of the monoclonal antibodies to so-called FC receptors of these non-T cells. This result is an important condition for the therapeutical application of “directly” stimulating CD28-specific monoclonal antibodies, wherein an artificial cross-linking with anti-immunoglobulin antibodies in the complete organism cannot be performed.

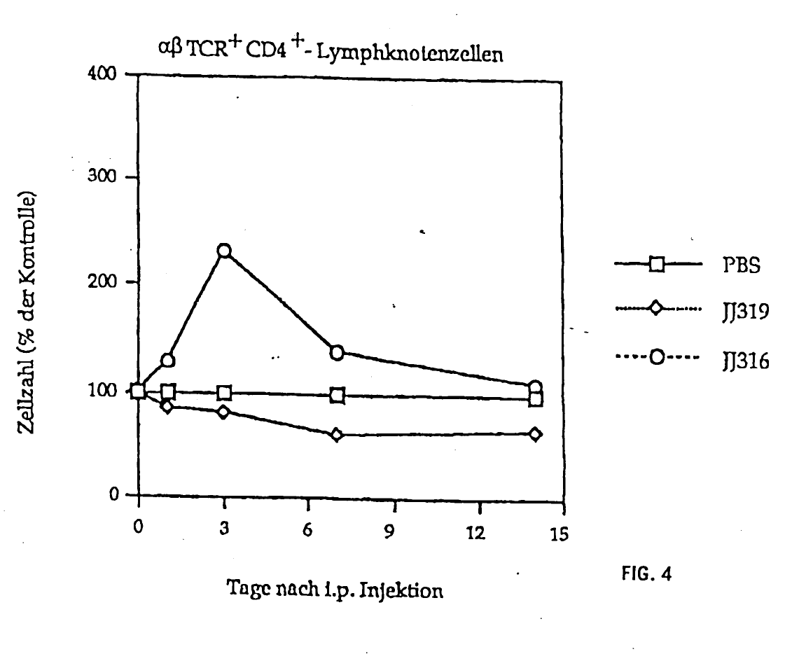

“Directly” activating CD28-specific monoclonal antibodies lead to an increase of the CD4 T cell count in the intact organism. FIG. 4 shows this for the lymph nodes of the rat having received per day 0.1 mg of the “directly” stimulating CD28-specific monoclonal antibody (JJ316) or of the “classic” CD28-specific monoclonal antibody (JJ319). With the monoclonal antibodies directly activating according to the invention with specificity for human CD28 and their capability to simulate the increase of the T lymphocytes count, analogous effects are achieved. In the present example the CD4 T cell count is only temporarily increased; the reason for this is that healthy animals were treated with normal CD4 T cell counts. The “excessive” cells due to proliferation stimulation were eliminated by homeostatic mechanisms.

The above results explained in detail show that an activation of the T lymphocytes takes place, and that without the necessity of further drugs.

### EXAMPLE 2

### Application of Antibodies Used According to the Invention in Conjunction With the HAART Therapy

2.1 General Information.

In the experiments described below, instead of the monoclonal antibody described in example 1, the antibody clone ANC28.1/5D10 of Ancell Corporation, USA, distributed by ALEXIS Deutschland GmbH, Giessener Str. 12, D.35305 Gruenberg, was used. It is known, with regard to these antibodies, that they induce IL-2 in DC28-positive cells. A proliferation-inducing activity of this antibody has however not yet been described. This antibody will be called in the following “aCD28”. In the following experiments, a monoclonal antibody not being directly stimulating, namely CD28.2, is used for comparison purposes.

The experiments were performed, unless otherwise stated, in human PBL or in PBL of rhesus monkeys. This cell culture sampling comes close to the situation of a complete organism, since the stimulating antibody is added in a dissolved manner, as provided for a therapy.

In all proliferation experiments, measurements were taken by a pulse of 1 μCi ³H thymidine for 16 hours between day 3 and day 4 and detection by means of a β detector.

Unless stated otherwise, cells were cultivated in 0.2 ml medium (RPMI 1640, available from GIBCO/BRL, containing 5% human AB serum) with a density of 1 million cells per ml in the gassed incubator.

2.2 Induction of T Cell Proliferation in Peripheral Blood Lymphocytes and/or Monocytes (PBMC) by aCD28.

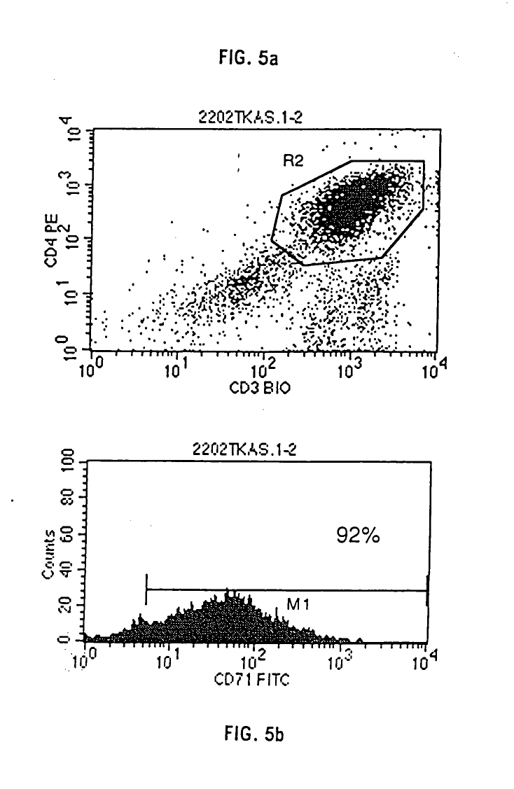

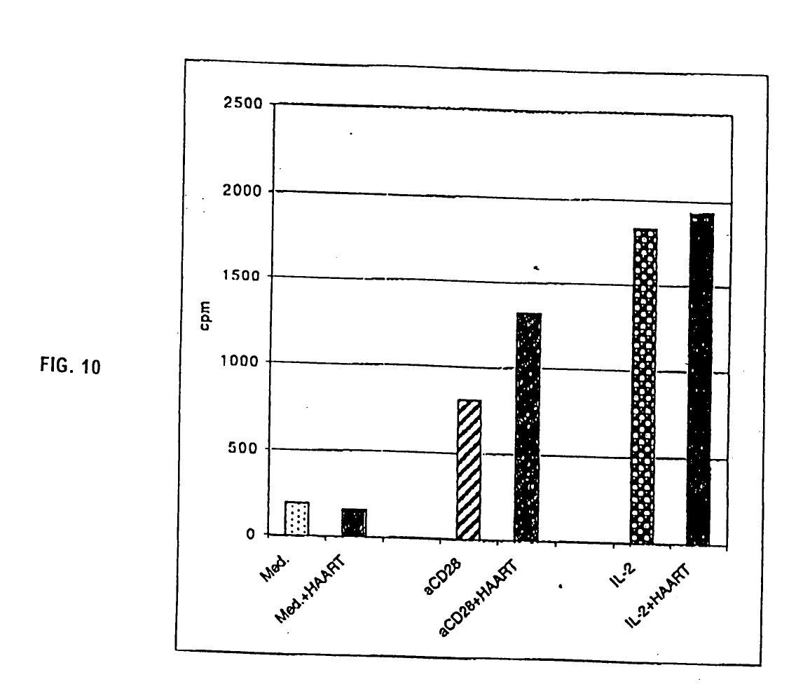

In FIG. 5 are shown experiments with regard to the extent of the activation of the proliferation of CD4 T cells. The detection takes place by immunofluorescence and flow cytophotometry (FACS) on day four after stimulation of the cells in vitro. FIG. 10a shows so-called “dotplots”, wherein the CD4 T cells are defined by a gate (R2) (they express CD3 and CD4). In FIG. 10b is shown in a histogram the coloration of these CD4 T cells with a monoclonal antibody against the transferrin receptor. The expression of this surface receptor characterizes proliferating cells. It can be seen that in a large part, namely >90%, of the CD4+ T lymphocytes the proliferation is (directly) induced by aCD28. The examinations were performed with 5 μg/ml aCD28.

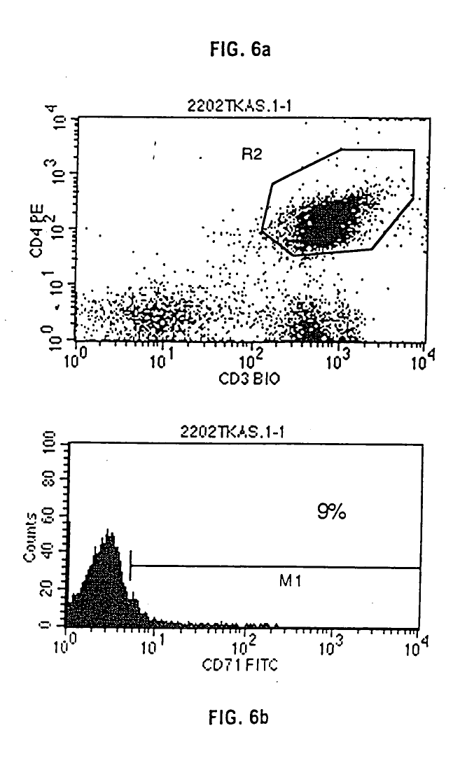

In FIG. 6 are shown corresponding representations, however without stimulation, as negative checks.

2.3: Comparison of the Stimulation of the Proliferation of Unseparated PBMC by aCD28 and IL-2.

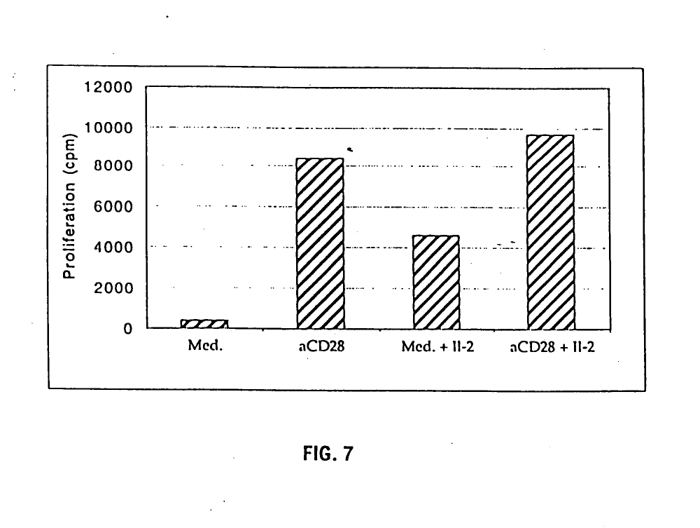

FIG. 7 shows that the proliferation of unseparated PBMC of not infected human donors, measured by the integration of ³H-marked thymidine, is stronger by aCD28 than by IL-2. This shows that more cells are addressed by means of aCD28 than by the in vivo therapy with HAART and IL-2 according to the state of the art. The cultivation was performed in the presence of 5 μg/ml aCD28 or 20 IU/ml IL-2 in 96-well round-bottom plates.

It is further important that IL-2 preferentially stimulates CD8 T lymphocytes for proliferation, whereas by aCD28 in particular the CD4 T cells are addressed. This can be seen from the data of table I: PBMC of an HIV-infected patient were stimulated for three or ten days with IL-2 or aCD28. By IL-2, not however by aCD28 resulted a distinct displacement in favor of CD8 T cells.

TABLE I

| | |
|---|---|
| after 3 days | after 10 days |

| | % CD4 | % CD8 | % CD4 | % CD8 |
|---|---|---|---|---|
| Medium | 51 | 49 | 50 | 50 |
| IL-2 | 46 | 54 | 27 | 73 |
| aCD28 | 57 | 43 | 62 | 38 |

2.4: Stimulation of the Proliferation of PBMC From Virus-Infected Organisms.

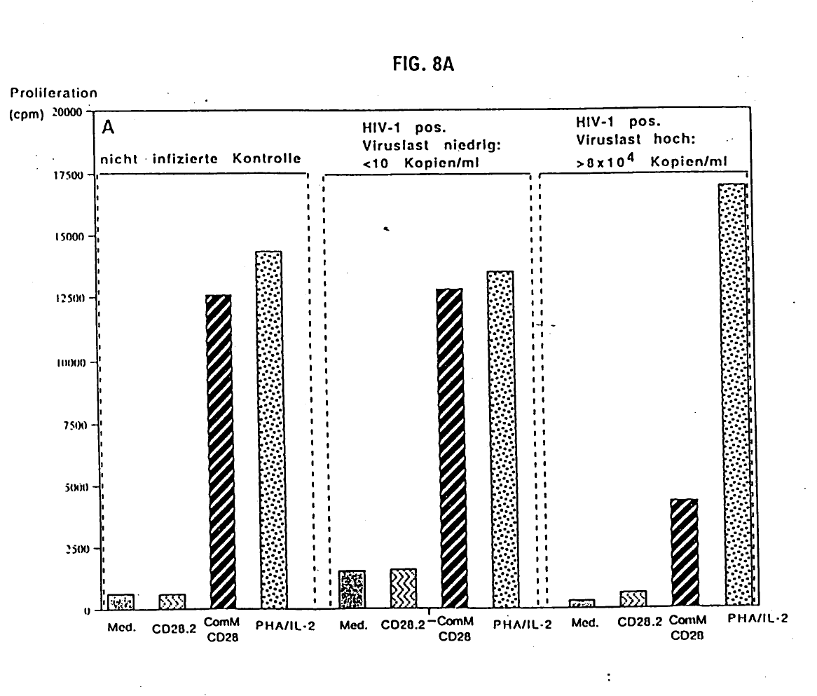

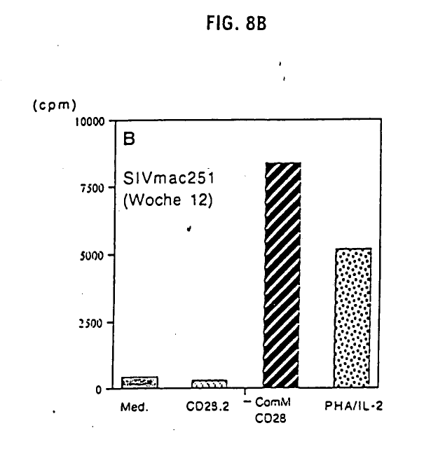

FIG. 8A shows the activating effect of aCD28 on the proliferation of PBMC of HIV-1-infected persons, FIG. 8B the corresponding effect of PBMC from HIV-infected rhesus monkeys. An amount of 5 μg/ml aCD28 was used. In all cases, strong proliferation could be induced, which was however weakest for massive HIV infection. The reason for this probably is the simultaneous massive induction of the virus count increase and the T cell destruction, which is prevented in a treatment according to the invention with common administration of HAART drugs (see also example 2.6a and FIG. 10).

2.5: Activation of the Proliferation in the Presence of HAART Drugs.

PBL's of an HIV-1-infected person were not treated and were treated with HAART drugs (zidovudine+didanosine+saquinavir; 0.1 μM) alone, with aCD28 (5 μg/ml) alone or with both. After six days, the proliferation was measured.

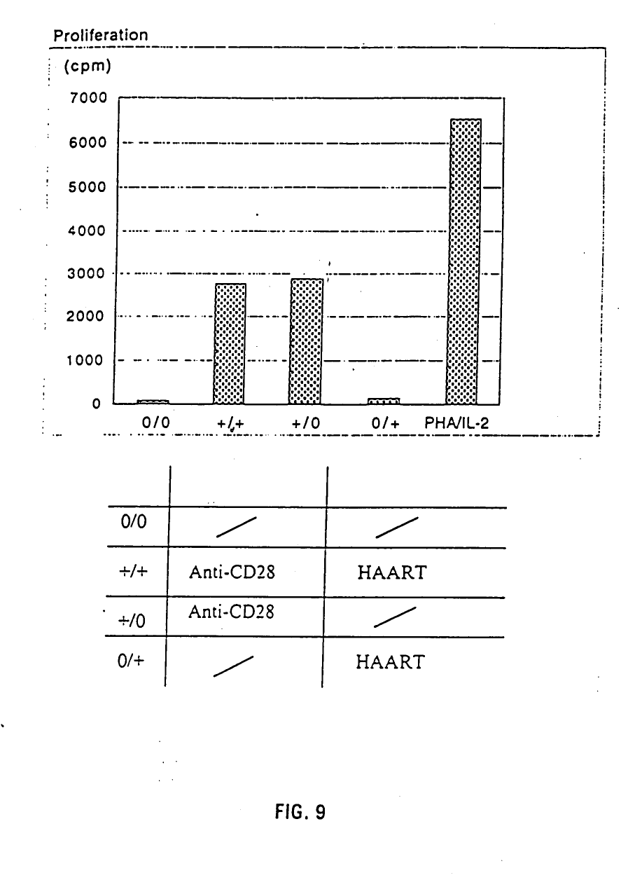

In FIG. 9 showing the results, it can be seen that the induction of the proliferation by means of aCD28 is not affected by the presence of HAART.

2.5a: Promotion of the CD28, Not However of the IL-2 Induced T Cell Proliferation.

PBMC of an HIV-1-infected patient were stimulated as described above. The proliferation was measured from day 3 to day 4. As sometimes detected with infected patients, IL-2 induces a stronger proliferation than CD28, mainly concerning however the CD8 cells not affected by HIV (see table I). This proliferation is not affected by HAART, whereas the aCD28-induced proliferation was improved by HAART (see FIG. 10).

2.6: Influence of aCD28/HAART on the Virus Replication.

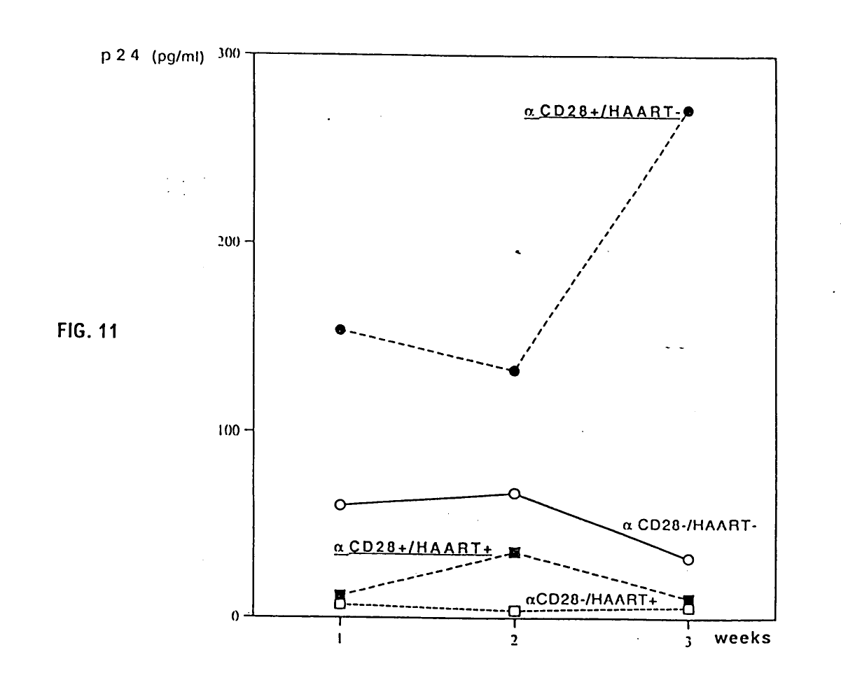

The virus replication in PBMC of HIV-1-infected persons was examined. In weekly intervals a free remainder was examined for the presence of p24Gag (virus protein). HAART was employed as in example 2.5. “+” means here and in the following the application of the respective drug, “−” absence. From FIG. 11 can be seen that the aCD28 stimulation also induces a massive virus production. With simultaneous HAART the virus production is however completely suppressed. PHA is phytohemagglutinin for comparison purposes.

2.7: Loss of the HIV-1 Production After Treatment With aCD28 and HAART.

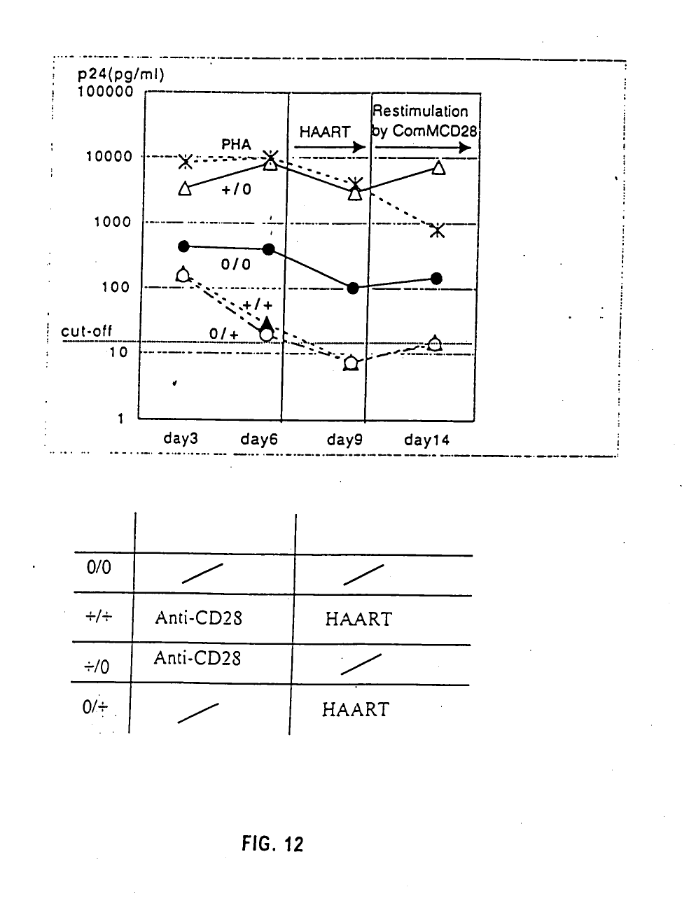

In FIG. 12 are shown the results of a test series, wherein PBMC of an HIV-1-infected person was treated until day 6 according to the table. From day 6 to day 9, a pure HAART treatment followed, and then a treatment with aCD28 (also called ComMCD28) from day 9 to day 14. The results confirm the ones from example 2.7, since according thereto even after renewed stimulation of cell cultures of HIV-infected PBMC, no virus production was detected after termination of the HAART therapy.

2.8: Loss of Pro-Viral HIV DNA, Too, After Treatment With aCD28 and HAART.

PBMC isolated from an HIV-1-infected person were cultivated according to the table and treated in vitro (drugs and drug amounts as in example 2.5). On the 11^{th} day of cultivation, an HIV-1-specific DNA-PCR was performed:

a) external; primer:

JA4 (gag1319-1338): gaa ggc ttt cag ccc aga ag
JA7 (gag1615-1596): tct cct act ggg ata ggt gg

Annealing temperature: 47° C.; 42 cycles.

b) nested; primer:

JA5 (gag1446-1465): acc atc aat gag gaa gct gc
JA6 (gag1577-1558): tat ttg ttc ctg aag ggt ac

Annealing temperature: 45° C.; 25 cycles.

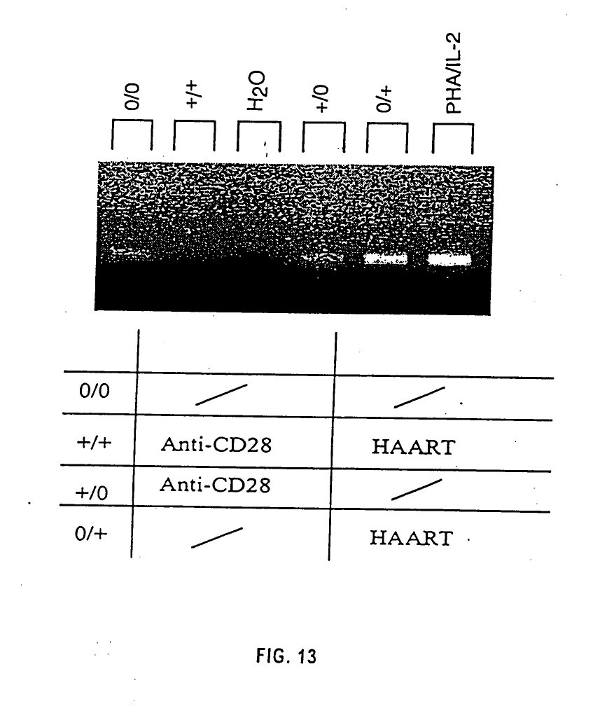

In FIG. 13 can be seen that pro-viral HIV DNA is not detectable for a combined treatment with HAART and monoclonal antibodies employed according to the invention, whereas in other cases of the table pro-viral HIV was always present. This demonstrates in a test system coming very close to the situation of the complete organism the successful destruction also of the reservoir of pro-viral structures by the invention.

We claim:

1. An application of monoclonal antibodies being specific for CD28 and activating T lymphocytes of several to all sub-groups without occupying an antigen receptor of the T lymphocytes and thus in an antigen-unspecific manner, or of an analogue thereto, for producing a pharmaceutical composition in an embodiment as a preparation or preparation package for treating virus infections of the human body or of body of a lower warm-blooded animal with infected T lymphocytes, wherein the pharmaceutical composition further contains a virus inhibitor.

2. An application according to claim 1, wherein CD4 T lymphocytes, in particular human CD4 T lymphocytes, are infected, and wherein the monoclonal antibodies are specific for human CD28 and the monoclonal antibodies are as an option well tolerated for human beings.

3. An application according to claim 1, wherein the virus infection is an infection caused by retrovirus, in particular lentivirus, for instance HIV.

4. An application according to claim 1, wherein the virus inhibitor is a reverse transcriptase inhibitor, preferably a pyrimidine nucleoside analogue, most preferably 3′-azido-3′-desoxythymidine (AZT or zidovudine), and as an option further other nucleoside analogues different therefrom, preferably 3TC, are included in the pharmaceutical composition.

5. An application according to claim 1, wherein the pharmaceutical composition further contains a protease inhibitor, and as an option further protease inhibitors different therefrom.

6. A pharmaceutical composition in an embodiment as a preparation or preparation package, with pharmaceutical effective doses of the following drug components:
a) preferably monoclonal antibodies well tolerated for human beings, preferably being specific for CD28, preferably human CD28 and activating T lymphocytes, preferably human T lymphocytes, of several to all sub-groups without occupying an antigen receptor of the T lymphocytes and thus in an antigen-unspecific manner, or an analogue thereto,
b) a virus inhibitor, preferably a reverse transcriptase inhibitor,
c) as an option a reverse transcriptase inhibitor being different from b),
d) as an option a protease inhibitor,
as an option a protease inhibitor different from d).

7. A pharmaceutical composition according to claim 6, wherein the drug components b) to e) are selected, dosed and made ready to administer according to the HAART therapy.

8. A pharmaceutical composition according to claim 6, wherein the drug components drug component b) is a pyrimidin nucleoside analogue, preferably 3′-azido-3′-desoxythymidine, and/or the drug component c) is 3TC, and/or the drug component d) is provided in a conclusive manner.

9. A pharmaceutical composition according to claims 6, wherein the drug components b) to e) are part of a first package component, and the drug component a) is part of a second package component.

10. A package component according to claims 6, containing the drug component a).

11. A method for treating virus infections with retrovirus, in particular lentivirus, for instance HIV, wherein the pharmaceutical composition according to claim 6 is administered to a human body or to the body of a lower warm-blooded animal attacked by the infection.

12. A method for treating virus infections with retrovirus, in particular lentivirus, for instance HIV, wherein the following drug components are administered to a human body or to the body of a lower warm-blooded animal in pharmaceutically effective doses:
a) preferably monoclonal antibodies well tolerated for human beings and being specific for CD28, preferably human CD28 and activating T lymphocytes, preferably human T lymphocytes of several or all sub-groups without occupying an antigen receptor of the T lymphocytes and thus in an antigen-unspecific manner, or an analogue thereto,
b) a virus inhibitor, preferably a reverse transcriptase inhibitor,
c) as an option a reverse transcriptase inhibitor different from b),
d) as an option a protease inhibitor,
e) as an option a protease inhibitor different from d).

13. A method according to claim 12, wherein the drugs b) to e) are selected, dosed and administered according to the HAART therapy and wherein the drug component a) is administered before, together with or after the drug components b) to e).

14. A method according to claim 123, wherein the drug components b) to e) are administered continuously and the drug component a) once or several times in time intervals with breaks.

15. A method according claim 12, wherein the drug component b) is a pyrimidine nucleoside analogue, preferably 3′-azido-3′-desoxythymidine, and/or
the drug component c) is 3TC, and/or
the drug component d) is provided in a conclusive manner.

16. An application of a preferably monoclonal antibody a) well tolerated for human beings and being specific for CD28, preferably human CD28 and activating T lymphocytes, preferably human T lymphocytes of several or all sub-groups without occupying an antigen receptor of the T lymphocytes and thus in an antigen-unspecific manner, or an analogue thereto, and a virus inhibitor b), preferably a reverse transcriptase inhibitor in the form of a mixture or of spatially separated compositions, one of those containing the drug component a) and the other one containing the drug component b), as a means for continuous and/or dis-continuous application during the treatment of virus infections with retrovirus, in particular lentiviruses, for instance AIDS, in human beings or lower warm-blooded animals according to a treatment plan comprising one or more cycles, the treatment plan comprising the following steps:
i) first the drug component b) is continuously administered in a pharmaceutically effective dose,
ii) after a given time duration of step i) the drug component a) is administered in a pharmaceutically effective dose, with continued administration of the drug component b),
iii) as an option step ii) will be repeated once or several times after a given break period, with continued administration of the drug component b).

17. An application according to claim 16, wherein the administration of the drug component b) takes place by a treatment sub-plan being the HAART therapy.

18. An application, pharmaceutical composition, package component or method according to claim 1, wherein the monoclonal antibody is CMY-2, available from hybridoma cells according to deposit DSM ACC2353, or the clone ANC28.1/5D10, or a preferably humanized or human-tolerated variant of the clone ANC28.1/5D10.
